(19) United States
(12) Patent Application Publication
(10) Pub. No.: US 2004/0115791 A1
(43) Pub. Date: Jun. 17, 2004

(54) Method and system of disposing food wastes

(76) Inventors: Suk-Ku Sim, Daejeon (KR); Eun-Kwan Lee, Kawngjoo-Kun (KR)

Correspondence Address: DINSMORE & SHOHL, LLP, 1900 CHEMED CENTER, 255 EAST FIFTH STREET, CINCINNATI, OH, 45202, US

(21) Appl. No.: 10/469,222

(22) PCT Filed: Feb. 27, 2002
(86) PCT No.: PCT/KR02/00329

(30) Foreign Application Priority Data
Feb. 27, 2001 (KR) 2001-09864

(51) Int. Cl. A01H 15/00; C12N 1/14

(52) U.S. Cl. 435/254.1; 800/297

(57) ABSTRACT

The present invention relates to a novel salt tolerant oyster mushroom strain Pleurotus ostreatus DH-1012; a method of producing a novel strain Pleurotus ostreatus DH-1012; a method of disposing food wastes using salt tolerant mushroom strains, in particular, a novel strain Pleurotus ostreatus DH-1012, comprising a food wastes pre-processing step, a mushroom cultivating step, and a specialty bio-feed and organic fertilizer, and manure producing step; a mushroom cultivated according to the method: and a specialty bio-feed and organic fertilizer, and manure produced according to the method. The present invention also relates to a system of disposing food wastes using salt tolerant mushroom strains, comprising a food wastes pre-processing system, a mushroom cultivating, and a specialty bio-feed and organic fertilizer, and manure producing system; a mushroom cultivated by the system; and a specialty bio-feed and organic fertilizer, and manure produced by the system.

### TECHNICAL FIELD

The present invention relates to a method and a system of disposing food wastes for making a cultivating compost with food wastes and cultivating edible mushrooms from the compost, and, for recycling the remaining mushroom compost after harvesting the mushrooms as a specialty additive, which is used for bio-feed and organic fertilizer, and manure.

And more particularly, the present invention relates to a salt tolerant oyster mushroom-strains, a method and a system of disposing food wastes using the mushroom strains, edible mushrooms cultivated by the method and the system, specialty additive for specialty bio-feed and organic fertilizer and manure produced by the method and the system, and a digital monitoring and control system which has a digital automatic central console for monitoring and controlling the whole system automatically.

### BACKGROUND ART

All over the world, the food waste increases every year due to the increase of population, the development of restaurant industry, the change in dining habits, and the development of economy. Especially, the food wastes which contain high content of water go bad quickly. When it is incinerated, its thermal efficiency is low and it generates dioxin that results in pollution of environment. When it is buried in a landfill, it becomes a main source of seepage water that pollutes ground and underground water as well as the soil.

Therefore, various methods for disposing such food wastes were proposed. Among the methods for disposing food wastes, the methods for recycling food wastes include fermenting it into fertilizer or dry feed, wet feed, incinerating it, and method of making it a compost for cultivating edible mushroom.

Even though there are differences in the characteristics and condition of the food wastes according to eating habits of each country, most of food wastes contain water and salt. And after the food wastes are fermented, the salt still remains in the processed fertilizer, harming plants and the soil. And other methods of disposing food wastes also cannot reduce the salt content in the food wastes. And the water in the food wastes pollutes soil and ground as well as underground water by seepage water.

For resolving these problems, the methods of eliminating salt content from the food wastes are known from the Korean patent No. 185264 issued to Cho Won-dae, Dec. 23, 1998, “Device for eliminating salt from food wastes,” and in the Korean patent No. 200031 issued to Lee Jae-mun, Mar. 8, 1998, “Compost fermenting agent using food wastes, a method of making the same, and a method of making stock waste water into compost using the agent.”

For eliminating salt from food wastes, the patent No. 185264 employs a centrifugal separation method, which separates water from the food wastes and then salt from the separated water. This method, however, cannot be adapted to solid food wastes and the centrifugal separation consumes too much energy.

For eliminating salt from food wastes, the patent No. 200031 employs a method of mixing a specified amount of calcium compounds with food wastes. The calcium compounds then react with NaCl in the food wastes and are converted into Na compounds such as NaSO₄, NaCO₃, and CaCl₂ which are water soluble compounds, so that there should be one more step, namely the step of eliminating water, to eliminate salt from food wastes. The above methods are not economical because they need extra physical or chemical treatment for eliminating salt.

The method of disposing food wastes by using it as a mushroom compost for cultivating edible mushroom has a critical problem that the usual mushrooms cannot grow well in the salty compost. So there is a need for a salt tolerant mushroom strain which can be cultivated in the salty compost.

As mentioned above, an environment-friendly method of recycling food wastes by reducing salt content in the food wastes without using extra physical or chemical treatment, and by re-using the salt-reduced food wastes that are economical and do not induce secondary environmental pollution, has long been desired in the art.

### DISCLOSURE OF INVENTION

Therefore, it is an object of the present invention to provide a novel salt tolerant strain that can solve the problems of the conventional methods of disposing food wastes.

It is another object of the invention to provide a method and system of disposing food wastes thereby in that edible mushrooms are cultivated with the salt tolerant mushroom strains which include the novel strain by using food wastes as a compost, and the remaining compost after harvesting the cultivated mushroom is used for producing specialty additive for specialty bio-feed and organic fertilizer and for producing manure.

It is still an object of the invention to provide a mushroom cultivated by the method and the system of disposing food wastes using the salt tolerant mushroom strain including the novel strain.

Also, it is an object of the invention to provide a specialty additive for the specialty bio-feed and organic fertilizer and manure produced by the method and the system of disposing food wastes using the salt tolerant mushroom strain including the novel strain. It is an object of the invention to eliminate stench by a bio-deodorizing system which uses deodorizing microorganism, and carriers, and by designing an airtight closed system from the feeding process of food wastes to the mixing process of dry cellulose for making a compost, while the stench is created during the feeding process.

It is still an object of the invention to provide an automatic system for handling a large quantity of food wastes, and to provide a central console for implementing digital automatic monitoring and control, display of monitoring and control variables, and internet monitoring to remotely monitor the system.

In order to accomplish the objects, the present invention provides a novel strain Pleurotus ostreatus DH-1012 (deposit No. KCTC 0938BP).

The present invention also provides a method of producing a strain Pleurotus ostreatus DH-1012, comprising the steps of:

step 1 of culturing numbers of hyphae with different concentration of salt in a compost and selecting a hypha of which the growing is less affected when salt concentration is higher;

step 2 of obtaining numbers of strains by separating monospore of the selected hypha in the aseptic condition and inducing mutation by ultraviolet rays irradiation;

step 3 of selecting an auxotroph strain which has fertility from the mutation induced strains;

step 4 of obtaining numbers of strains by selecting one from high quality oyster mushrooms varieties, then by separating monospores from the selected one, and inducing mutation by ultraviolet rays irradiation;

step 5 of selecting auxotroph strain which has fertility from the mutation induced strains; and

6 step of producing somatic cell hybrid strains by monospore-mating the strain selected in the step 3 with the strain selected in the step 5.

The present invention also provides a method of producing a novel strain Pleurotus ostreatus DH-1012, wherein the strain used in the step 1 is Pleurotus ostreatus, and the selection comprise culturing of hyphae in the PDA composts of which salt concentration is increased by 0.5% in the range of 0-3% and selecting a hypha that is less affected when the salt concentration is higher, and the strain used in the step 4 is Pleurotus ostreatus Nonggi 2-1.

The present invention also provides a method of disposing food wastes using salt tolerant mushroom strains, comprising the steps of:

food wastes pre-processing step for forming a compost that is suitable for cultivating mushroom; and

mushroom cultivating step for inoculating the salt tolerant mushroom strains in the compost, then culturing and cultivating mushroom.

The food wastes pre-processing step comprises the steps of:

step 1 of eliminating foreign objects from the carried-in food wastes;

step 2 of crushing the separated food wastes into a predetermined size; and

step 3 of deodorizing the crushed food wastes, wherein these steps can be proceeded sequentially or simultaneously.

The mushroom cultivating step comprises the steps of:

step 1 of forming a compost by modulating the water content of the separated and crushed food wastes using dry cellulose;

step 2 of inserting a predetermined amount of the formed compost into a cultivating vessel;

step 3 of sterilizing the compost in the vessel;

step 4 of cooling the sterilized compost and then inoculating the salt tolerant mushroom strains into the compost in the vessel;

step 5 of culturing the inoculated salt tolerant mushroom strains; and

step 6 of cultivating the mushroom of the compost in the vessel and then harvesting it.

The present invention also provides a method of disposing food wastes using salt tolerant mushroom strains, comprising the steps of:

food wastes pre-processing step for forming a compost that is suitable for cultivating mushroom; and mushroom cultivating step for inoculating the salt tolerant mushroom strains in the compost, then culturing and cultivating mushroom, and further comprising the step of specialty additive producing step for producing specialty additive, which is used for specialty bio-feed and organic fertilizer, with the remaining compost after harvesting the cultivated mushroom.

The specialty additive producing step comprises the steps of:

step 1 of separating the remaining compost after harvesting mushrooms from the compost vessel;

step 2 of crushing the separated compost into a predetermined size:

step 3 of eliminating foreign objects from the crushed compost;

step 4 of producing specialty additive that contains mushroom hyphae by drying the foreign objects eliminated compost.

The present invention also provides a method of disposing food wastes using salt tolerant mushroom strains, comprising the steps of: food wastes pre-processing step for forming a compost that is suitable for cultivating mushroom; and

mushroom cultivating step for inoculating the salt tolerant mushroom strains in the compost, then culturing and cultivating mushroom, and further comprising the step of manure producing step for producing manure with the remaining compost after harvesting the cultivated mushroom.

The manure producing step comprises:

step 1 of separating the remaining compost after harvesting mushrooms from the compost vessel;

step 2 of crushing the separated compost into a predetermined size; and

step 3 of eliminating foreign objects from the crushed compost.

The manure producing step may further comprise a step of drying the foreign objects eliminated compost.

The manure produced in said step can be used as manure itself and can also be used by mixing with other substances such as conventional manure. The salt content of manure can be adjusted by mixing with other substances.

The present invention also provides a method of disposing food wastes using a salt tolerant strain, comprising the steps of food wastes preprocessing step (a), mushroom cultivating step (b), and specialty additive producing step (c), in which:

the waste pre-processing seep (a) comprises the steps of eliminating of foreign metallic objects from the food wastes, crushing of the separated food wastes into a predetermined size, and deodorizing of the crushed food wastes; wherein

the mushroom cultivating step (b) comprises the steps of:

forming of the compost by mixing dry cellulose so that the water content of the food wastes is 50 to 70%;

inserting of a predetermined amount of the compost into the cultivating vessel;

sterilizing of the compost by steam at 60° C. to 121° C. that is supplied for 1 to 5 hours;

cooling of the compost down to 20 to 25° C.;

inoculating of a predetermined amount of the novel strain Pleurotus ostreatus DH-1012 into the compost;

culturing of the strain for 15 to 30 days in an aseptic culturing room at 20 to 25° C.;

growing of the cultured strain for about 10 days at 8 to 18° C.; and

harvesting of the mushroom; and wherein the specialty additive producing step (c) comprises the steps of:

separating of the remaining compost after harvesting mushrooms from the compost vessel;

crushing the separated compost into a predetermined size;

eliminating of foreign objects from the crushed compost; and

producing of specialty additive by drying of the separated compost so that the water content of the medium is 10 to 15%.

The present invention also provides a method of disposing food wastes using a salt tolerant strain, comprising the steps of food wastes pre-processing step (a), mushroom cultivating step (b), and manure producing step (d), in which:

the waste pre-processing step (a) comprises the steps of eliminating of foreign metallic objects from the food wastes, crushing of the separated food wastes into a predetermined size, and deodorizing of the crushed food wastes; wherein

the mushroom cultivating step (b) comprises the steps of:

forming of the compost by mixing dry cellulose so that the water content of the food wastes is 50 to 70%;

inserting of a predetermined amount of the compost into the cultivating vessel;

sterilizing of the compost by steam at 60° C. to 121° C. that is supplied for 1 to 5 hours;

cooling of the compost down to 20 to 25° C. inoculating of a predetermined amount of the novel strain Pleurotus ostreatus DH-1012 into the compost;

culturing of the strain for 15 to 30 days in an aseptic culturing room at 20 to 25° C.;

growing of the cultured strain for about 10 days at 8 to 18° C.; and

harvesting of the mushroom; and wherein the manure producing step (d) comprises the steps of:

separating of the remaining compost after harvesting mushrooms from the compost vessel;

crushing the separated compost into a predetermined size; and

eliminating of foreign objects from the crushed compost.

The present invention provides a mushroom cultivated by the method of disposing food wastes.

The present invention also provides specialty additive for bio-feed and organic fertilizer produced by the method of disposing food wastes.

The present invention also provides specialty bio-feed and organic fertilizer produced by mixing with the specialty additive produced by the method of disposing food wastes. The present invention also provides manure produced by the method of disposing food wastes.

The present invention also provides a system of disposing food wastes using salt tolerant mushroom strains which comprises a food wastes pre-processing system for forming a compost suitable for mushroom cultivating, and a mushroom cultivating system for cultivating mushrooms by inoculating salt tolerant mushroom strains into the compost and culturing them.

The present invention also provides a system of disposing food wastes using salt tolerant mushroom strains which further comprises a specialty additive producing system for producing specialty additive for bio-feed and organic fertilizer with the compost remaining in the compost vessel after harvesting the mushroom.

The present invention also provides a system of disposing food wastes using salt tolerant mushroom strains which further comprises a manure producing system for producing manure with the compost remaining in the compost vessel after harvesting the mushroom.

The present invention also provides a system of disposing food wastes using salt tolerant mushroom strains, wherein the salt tolerant oyster mushroom strain is a novel strain Pleurotus ostreatus DH-1012.

The food wastes pre-treating system in accordance with the invention comprises an entrance through which the collected food wastes enter and an exit through which the food wastes are discharged. Between the entrance and the exit, there are a fixed blade which is fixed to the frame and which has a plurality of tooth-form blades and a rotating blade which is installed with a predetermined gap from the fixed blade. At least one crusher for crushing the food wastes into a predetermined size by the rotation of the rotating blade and at least one foreign object eliminating means for eliminating metallic foreign objects from the food wastes are installed in the pre-processing system. The crusher and the foreign object eliminating means are designed air-tight closed systems, and the pre-processing system communicates with at least one bio-deodorizing system via stench gas suction pipes.

The mushroom cultivating system comprises:

at least one compost forming means (a) for forming a compost by adjusting the water content in the crushed and separated food wastes;

at least one compost inserting means (b) for inserting a predetermined amount of the formed compost into a cultivating vessel;

at least one sterilizing means (c) for sterilizing the compost inserted in the cultivating vessel;

at least one strain inoculating means (d) for cooling the sterilized compost and inoculating salt tolerant mushroom strains into the compost;

at least one hypha culturing means (e) for culturing the inoculated salt tolerant mushroom strains; and

a mushroom cultivating means (f) for cultivating mushroom with the cultured hypha.

The compost forming means (a) comprises a transporting means for transporting the food wastes which are pre-processed in the food wastes pre-processing system, and a mixer for mixing dry cellulose with the food wastes that is transported by the transporting means.

The compost inserting means (b) comprises a transporting means for transporting the compost formed by the compost forming means, a supplying means for supplying the cultivating vessel for inserting the transported compost, and an inserter for inserting the transported compost into the supplied cultivating vessel.

The compost sterilizing means (c) comprises a transporting means for transporting the compost inserted cultivating vessel, and a sterilizer for sterilizing the transported cultivating vessel.

The strain inoculating means (d) comprises a transporting means for transporting the cultivating vessel into which the sterilized compost is inserted, a means for cooling the vessel into which the transported compost is inserted and maintaining the aseptic condition, and an inoculater for inoculating a predetermined amount of salt tolerant mushroom strains into the cooled compost.

The hypha culturing means (e) comprises a transporting means for transporting the strain inoculated cultivating vessel, and a cooler/heater, a humidifier, a means of illumination, and a ventilation system for making the optimum culturing condition for the inoculated mushroom strains in the transported cultivating vessel.

The mushroom cultivating means (f) comprises a transporting means for transporting the cultured hypha, and a cooler/heater, a humidifier, a means of illumination, and a ventilating system for making the optimum culturing condition for the cultured mushroom hypha in the transported cultivating vessel.

The specialty additive producing system of the present invention comprises:

a compost separating means (a) for separating the compost from the cultivating vessel after the mushrooms are harvested;

a crushing means (b) for crushing the separated compost into a predetermined size;

a foreign objects eliminating means (c) for eliminating the foreign objects from the crushed compost;

a drying means (d) for drying the foreign objects eliminated compost; and

a packing means (e) for packing the dried specialty additive into the predetermined size.

The compost separating means (a) comprises a transporting means for transporting the cultivating vessel in which only the compost is left after the cultivated mushrooms are harvested, a separator for separating the compost from the transported cultivating vessel, and a storing tank for storing the separated compost.

The foreign objects eliminating means (c) comprises a transporting means for transporting the crushed compost, an eliminating means for eliminating foreign objects from the transported compost, and a discharging means for discharging the separated foreign objects.

The driving means (d) comprises a transporting means for transporting the foreign objects eliminated compost to a feeding hopper, an inputting means for inputting a predetermined amount of the transported compost to a dryer, an indirect drying means using heater or steam from a boiler or direct drying means using a hot air blower.

The packing means (e) comprises a transporting means for transporting the specialty additive crushed into the predetermined size by the crushing means, a storing means for storing the transported specialty additive, a providing means for providing a packing case for packing the stored specialty additive, and a packing device for packing the specialty additive which is transported from the storing means in a predetermined amount into the packing case which is provided by the providing means.

The manure producing system of the present invention comprises:

a compost separating means (a) for separating the compost from the cultivating vessel after the mushrooms are harvested;

a crushing means (b) for crushing the separated compost into a predetermined size; and

a foreign objects eliminating means (c) for eliminating the foreign objects from the crushed compost.

The compost separating means (a) comprises a transporting means for transporting the cultivating vessel in which only the compost is left after the cultivated mushrooms are harvested, a separator for separating the compost from the transported cultivating vessel, and a storing tank for storing the separated compost.

The foreign objects eliminating means (c) comprises a transporting means for transporting the crushed compost, an eliminating means for eliminating foreign objects from the transported compost, and a discharging means for discharging the separated foreign objects.

The present invention provides a system of disposing food wastes using salt tolerant mushroom strains, in which the transporting means in the system is a horizontal belt conveyor, wherein the horizontal belt conveyor is made of an elastic material so that it is stretched by the weight of the material transported by the conveyor and both ends of the conveyor are maintained at a predetermined height by a shape fixing guide, and wherein the conveyor is a screw conveyor having an axis of rotation operated by the power of a transporting motor and spiral blade members fixed to the axis of rotation.

The present invention also provides a system of disposing food wastes using salt tolerant mushroom strains which further comprises a digital monitoring and control system for automatically monitoring and controlling the system of pre-processing food wastes, the system of cultivating mushroom, the system of producing specialty additive, and the system of producing manure.

The digital monitoring and control system comprises:

a central console for monitoring and controlling the system of pre-processing food wastes, the system of cultivating mushroom, the system of producing specialty bio-feed and organic fertilizer, processes performed by the systems and products produced by the systems through the input/output and transmission/reception of various monitoring and control data;

monitoring and control boards installed in the systems and implemented through the central control console;

sensors installed in each part of the systems and controlled by the control board; and

a communication interface means performing transmission/reception of data between the central control console and the sensors and local control boards.

The present invention also provides mushrooms cultivated by the system of disposing food wastes.

The present invention also provides specialty additive produced by the system of disposing food wastes.

The present invention also provides specialty bio-feed and organic fertilizers produced by mixing with the specialty additive produced by the system of disposing food wastes.

The present invention also provides manure produced by the system of disposing food wastes.

The present invention is characterized in using a mushroom strain, which decomposes organic materials in the natural world so as not to induce the secondary pollution of environment and for environment-friendly treatment maintaining the natural cycle of an ecological system, and to preserves the nature, using a characteristic of food wastes with high content of water and salt.

First, mushrooms belong to fungi, but are called higher fungi because they have a fruit body unlike other fungi. Mushrooms produce other products by using organic materials such as food wastes as a compost. A mushroom needs water as much as 70% when it grows, and because food wastes of Korea and China contain high content of water in it, it is suitable for growing mushroom, and by using food wastes in this way the secondary environmental pollution can be prevented.

Next, in general, mushrooms can not grow well in a salty compost, so that salt tolerant mushroom strains should be selected.

A novel strain can be selected in nature by chance, but in general the artificial techniques such as monospore mating, mutation inducement, protoplasm fusion, gene recombination are used.

The problems that rise in breeding mushroom by monospore mating are incompatibility in the same line, and impossibility of hybridization between the species of remote types. The protoplasm fusion is the one that complements the weak point, and generally the auxotroph strains are used therefore. When they are fused, the auxotroph that cannot be raised in a minimum compost can be raised by complementation. The auxotroph strains that are impossible to be raised due to a defect of enzyme activation in a specific base sequence during metabolism are recovered in the defect of enzyme activation by fusion by inner cell nucleus coexistence or DNA insertion into chromosomes. The selection of a protoplasm fusion strain consists of raising it in the minimum compost, and the comparison of fungi cluster form of the parent strain used in the fusion makes it possible to distinguish it more clear. Even though a hybridized somatic cell is obtained by overcoming incompatibility by protoplasm fusion, a mushroom fruit body may not be induced. Therefore, forming a fruit body is a field that should be further studied and the problem that should be overcome in the breeding of somatic cells hybridized between far-related species by protoplasm fusion. The gene recombination is a recently issued social problem about genetically modified organisms (GMOs). So in the present invention salt tolerant mushroom strains are produced by monospore mating and mutation inducement. And the newly produced strain was named Pleurotus ostreatus DH-1012, and deposited to Korea Research Institute of Bioscience and Biotechnology (KRIBB), and given the deposit No.

KCTC 0938BP, dated from Jan. 9, 2001.

The present invention also provides a method and a system of disposing food wastes using salt tolerant mushroom strains comprising a novel strain Pleurotus ostreatus DH-1012.

The method of disposing food wastes according to the present invention is illustrated in FIG. 1 and comprises 3 steps, and also a system for embodying the method is provided.

Step 1 is a pre-processing process of food wastes for forming a compost suitable for cultivating mushroom, in which food wastes are fed and metallic foreign objects in the food wastes are eliminated and then the food wastes are crushed, then transported to a mixer for forming the compost.

Step 2 is a mushroom cultivating process for cultivating mushroom by inoculating salt tolerant mushroom strains into the compost and culturing them, in which the crushed food wastes transported to the mixer are mixed with dry cellulose to adjust water content, then is inserted in a cultivating vessel, the vessel is capped then sterilized and cooled, then salt tolerant mushroom strains, for example the novel strain Pleurotus ostreatus DH-1012 is inoculated and cultured. After the cultivated mushrooms are harvested the mushroom cultivating vessel is transported to a vessel separator.

Step 3 is a process of producing specialty additive with the remaining compost after the cultivated mushrooms are harvested, in which the remaining compost is separated from the vessel transported to the cultivating vessel separator, then dried, and then foreign objects in the compost are eliminated. Then the compost is crushed and then packed, so that the specialty additive containing mushroom strains is produced.

The present invention also provides a system of disposing food wastes comprising a system of pre-processing food wastes for forming a compost suitable for mushroom cultivating, a system of cultivating mushroom for inoculating salt tolerant mushroom strains into the compost, then culturing and cultivating mushroom, and a system of producing specialty additive for producing specialty additive with the remaining compost after harvesting the cultivated mushroom.

At first, as an example, the system of pre-processing food wastes for forming a suitable compost for cultivating mushroom is disclosed, as illustrated in FIG. 3.

In the pre-processing system, the metallic foreign objects is eliminated by using the metallic foreign objects eliminator (12) fixed to the rotating conveyor during transporting wet and salty food wastes fed through the feeding tank (10) by conveyor (11), then the foreign objects eliminated food wastes are crushed into a predetermined size by a crusher (13). To eliminate stench generated in the pre-processing system, the pre-processing system is designed airtight and the multi-stage bio deodorizing system (4) for eliminating stench gas by passing it through deodorizing microorganisms and carrier is used.

The multi-stage bio deodorizing system is, for example, disclosed in the Korean patent application No.2000-0070166 that is filed by the applicant of the invention.

The system of pre-processing food wastes is, for example, disclosed in the Korean patent application Nos. 2000-70164, 2000-70165, and 2000-70166., dated from Nov. 24, 2000.

In addition, the mushroom cultivating system for inoculating mushroom strains into the compost, culturing and cultivating them comprises:

at least one medium forming means (a) for forming a compost by adjusting the water content of the crushed and separated food wastes;

at least one compost inserting means (b) for inserting a predetermined amount of the compost into the cultivating vessel;

at least one sterilizing means (c) for sterilizing the compost inserted in the cultivating vessel;

at least one strain inoculating means (d) for cooling the sterilized compost and inoculating salt tolerant mushroom strains into the compost;

at least one hypha culturing means (e) for culturing the inoculated salt tolerant mushroom strains; and

at least one mushroom cultivating means (f) for cultivating mushroom with the cultured hypha.

The compost forming means (a) comprises a transporting means for transporting the food wastes that are pre-processed in the food wastes pre-processing system, and a mixer for mixing dry cellulose with the food wastes that is transported by the transporting means.

The compost inserting means (b) comprises a transporting means for transporting the compost formed by the compost forming means, a supplying means for supplying the cultivating vessel for inserting the transported compost, and an inserter for inserting the transported compost into the supplied cultivating vessel.

The compost sterilizing means (c) comprises a transporting means for transporting the compost inserted cultivating vessel, and a sterilizer for sterilizing the transported compost in the cultivating vessel.

The strain inoculating means (d) comprises a transporting means for transporting the cultivating vessel into which the sterilized compost is inserted, a means for cooling the vessel into which the compost transported by the transporting means is inserted and maintaining the aseptic condition, and an inoculater for inoculating a predetermined amount of salt tolerant mushroom strains into the cooled compost.

The hypha culturing means (e) comprises a transporting means for transporting the strain inoculated cultivating vessel, and a cooler/heater, a humidifier, a means of illumination, and a ventilating system for making the optimum culturing condition for the inoculated salt-tolerant mushroom strains in the transported cultivating vessel. The mushroom cultivating means (f) comprises a transporting means for transporting the cultured hypha, and a cooler/heater, a humidifier, a means of illumination, and a ventilating system for making the optimum culturing condition for the cultured salt-tolerant mushroom hypha in the transported cultivating vessel.

Hereinafter, the method and the system of cultivating mushroom are described in more detail.

At first, a compost suitable for mushroom cultivating is formed by mixing dry cellulose sources such as dry sawdust, waste cotton, rice-straw, etc. with the crushed food wastes transported by the transporting means so as to adjust the water content of the food wastes to be 50 to 70%.

Next, the formed compost is transported to an automatic bottling machine by the transporting means, and when a plastic container containing about 16 to 36 cultivating vessels that are plastic bottles is provided to the automatic bottling machine, the mixed compost is automatically filled into the bottle by the automatic bottling machine and the bottles are automatically capped by an automatic capping means.

Next, the compost in the plastic bottle is transported to the sterilizer equipped with a boiler by the transporting means, then is sterilized for 1 to 5 hours by steam at 0.0 to 1.0 kgf/cm² and 60 to 121° C. so that all the microorganisms in the compost are sterilized.

Next, when the sterilized compost is transported by the transporting means to the strain inoculating means, the compost is cooled to the room temperature of 20 to 25° C. in an aseptic room, then a salt tolerant mushroom strain, for example, the novel strain Pleurotus ostreatus DH-1012 is inoculated automatically into the compost in the bottle by an automatic inoculating machine.

Next, the cultivating vessel-into which salt tolerant mushroom strains are inoculated is transported to a strain culturing means by a transporting means, then the mushroom hyphae are cultured in the optimum culturing environment for about 15 to 30 days, wherein the optimum environment is obtained by maintaining the temperature of 20 to 25° C., adjusting humidity, and ventilating by a culturing means having a cooler/heater, a humidifier, a means of illumination, and a ventilating system.

Next, the cultivating vessel containing the cultured strains is transported to the mushroom cultivating means by a transporting means, then the mushrooms are cultivated in the optimum mushroom cultivating environment for about 10 days and then harvested, wherein the optimum environment is obtained by maintaining the temperature of 8 to 18° C., adjusting intensity of illumination and ventilating by a cultivating means having a cooler/heater, a humidifier, a means of illumination, and a ventilating system.

The specialty additive producing system comprises a compost separating means (a) for separating the compost from the cultivating vessel after harvesting mushrooms,

a crushing means (b) for crushing the separated compost into a predetermined size,

a foreign objects eliminating means (c) for eliminating foreign objects from the crushed compost,

a drying means (d) for drying the foreign objects eliminated compost, and a packing means (e) for packing the dried specialty additive.

The compost separating means (a) comprises a transporting means for transporting the cultivating vessel in which only the compost is left after the cultivated mushrooms are harvested, a separator for unbottling the compost from the transported cultivating vessel, and a storing tank for storing the unbottled compost.

The foreign objects eliminating means (c) comprises a transporting means for transporting the crushed compost, an eliminating means for eliminating foreign objects from the transported compost, and a discharging means for discharging the separated foreign objects.

The drying means (d) comprises a transporting means for transporting the foreign objects eliminated compost to a feeding hopper, an inputting means for inputting the transported compost into a dryer in a predetermined amount, an indirect drying means using heater or steam from a boiler or direct drying means using a hot air blower.

The packing means (e) comprises a transporting means for transporting the specialty additive, crushed into a predetermined size by the crushing means, a storing means for storing the transported specialty additive by a transporting means, a providing means for providing a packing case for packing the stored specialty additive, and a packing device for packing the specialty additive which is transported from the storing means in a predetermined amount into the packing case which is provided by the providing means.

Hereinafter, the system of producing specialty additive with the remaining compost after harvesting mushrooms is described in more detail by referring to FIG. 4.

At first, the transported compost that contains mushroom hyphae remaining after harvesting mushrooms is taken-out from the cultivating vessel by automatic unbottling machine (not shown) and is stored in the storing tank (30).

Next, a predetermined amount of the taken-out and stored compost is transported to the crusher (35) and crushed into a uniform size.

Next, for separating foreign objects such as vinyl, plastics, nonferrous metals etc. and other large foreign objects in the compost, the crushed compost is transported to the transporting means (31) and the foreign objects is eliminated by a foreign objects separator (34), for example, a shaking or rotating screen.

Next, the separated compost is dried by indirect heating using heater or steam, or by direct heating using a hot air blower so that the water content is lowered to 10 to 15% so that the specialty additive containing mushroom hyphae is produced.

Next, the produced specialty additive is transported to a storing tank (36) by the transporting means (31), then packed by the packing case providing means and packing means (not shown), then carried out.

The transporting means for the system is a horizontal belt conveyor, wherein the horizontal belt conveyor is made of elastic material so that it is stretched by the weight of the material transported by the conveyor while the both ends of the conveyor are maintained at a predetermined height by the shape fixing guide, and wherein the conveyor is a screw conveyor having an axis of rotation operated by the power of a transporting motor and spiral blade members fixed to the axis of rotation.

The present invention also provides a system of disposing food wastes using salt tolerant mushroom strains, which further comprises a digital monitoring and control system for monitoring and automatically controlling the system of disposing food wastes.

The digital monitoring and control system comprises:

a central console for monitoring and controlling the system of pre-processing food wastes, the system of cultivating mushroom, the system of producing specialty additive, the system of producing manure processes performed by the systems and products produced by the systems through the input/output and transmission/reception of various monitoring and control data;

sensors and local control boards installed in each part of the systems and linked to the central consol by Ethernet; and

communication interface means, i.e., Ethernet, perform transmission/reception of the data between the central console and the local control boards or sensors.

Hereinafter the automatic digital monitoring and control system is described in more detail by referring to the FIG. 5. The automatic digital monitoring and control system comprises the system of pre-processing food wastes (1), the system of cultivating mushroom (2), the system of producing specialty additive and manure (3), and the digital monitoring and control system (4), and each system is automatically monitored and controlled by the central console (5).

At first, the local control board which automatically controls the system of pre-processing food wastes (1) is equipped in the process room for pre-processing food wastes, and controls in situ by the on/off switch the amount of food wastes fed, the water content of food wastes, operation of the screw of the food wastes feeding tank, operation of the metallic foreign objects eliminator, operation of the conveyor which transports food wastes to the crusher, operation of the crusher, operation of the conveyor which transports food wastes from the crusher to a mixer.

The amount of food wastes fed, the water content, the temperature inside and outside of the process room are sensed by the sensors, and the sensed signals are A/D converted in a local processor and signaled by a PC processor, then input to the central console (5) by Ethernet (41) communication. The automatic bio-deodorizing system is couple to and controlled by the central console (5).

The operation of the mixer, the bottling machine, the capping machine, the boiler, the sterilizer, the inoculator, and the unbottling machine are performed by On/Off switch attached to each machine, and the vapor temperature, pressure, and operating time of the sterilizer are sensed by the sensors and the sensed signals are A/D converted in the local processor and signaled by the PC processor, then input to the central console (5) by Ethernet (41) communication.

The bundle of sensors that sense temperature, humidity, illumination, CO, concentration that show a ventilation state are equipped in many places of the culturing room and the growing room, and these sensors are each connected to the cooler/heater, humidifier, means of illumination, and ventilation system respectively so that the system is automatically controlled and that the temperature, humidity, illumination, and the ventilation in the culturing room and the growing room are maintained uniform, so that the high quality and high yield of mushrooms crops can be achieved.

In the system of producing specialty additive (3), foreign objects such as vinyl, nonferrous metals, and plastics in the compost are eliminated by the separating means such as a shaking or rotating screen, then the specialty additive is produced after crushing it into a uniform size. The amount of the remaining compost after harvesting mushroom is manually input and the water content of the compost is input by a sensor, then and input amount/hour to the dryer (32) is controlled according to the water content of the resultant specialty additive, and the dried specialty additive is discharged after specified time.

All the operations of the specialty additive producing system are performed by the On/Off switch equipped in the system, but the amount of food wastes input, water content, input/hour, and the quantity of specialty additive, etc. are sensed by a sensor, and the sensed signals are A/D converted in the local processor and signaled by the PC processor, then input to the central console (5) by Ethernet (41) communication. For the process control for mushrooms, a barcode is attached to the basket for transporting mushroom cultivating bottles so that the mushroom cultivating process of each basket can be traced, and the barcode is read by the barcode reader (17, 22, 23) which is equipped in the entrance of culturing means (20) and the entrance and exit of cultivating means (21), and the read data can be utilized in automatic control of mushroom cultivating and harvesting.

For implementing the digital automatic monitoring and control system, all the signals sensed by the sensors are A/D converted in the local processor and signaled by PC processor (42), then input to the central console (5) by Ethernet (41) communication.

The central console (5) receives an input from the sensors as shown in FIG. 5, analyzes and processes the signals, and monitors or automatically controls the system and the automatic control output is controlled by an actuator in the central console (5) through Ethernet (41) communication.

As shown in FIG. 6, input to central console (5) is controlled by the keyboard (50), the touch screen (52), or the mouse (51), and the main functions are the automatic control of the system, the monitoring using alarm sound and LEDs when a signal exceeds the set point in an emergency or abnormal running, calling of an operator and a plant supervisor in an emergency or abnormal running, remotely monitors a plant state through the internet, display of control variables as digital mechanism and graphs in screen by using a PC.

Such automatic digital monitoring and control of the system by the central console has merits of disposing food wastes in large quantities, improvement of quality and production of mushroom and specialty additive.

### BRIEF DESCRIPTION OF THE DRAWINGS

These and other features, aspects, and advantages of the present invention will help better understanding with regard to the following description, appended claims, and accompanying drawings, in which like components are referred to by like reference numerals. In the drawings:

FIG. 1 is a block diagram of a process of disposing food wastes using salt tolerant mushroom strains.

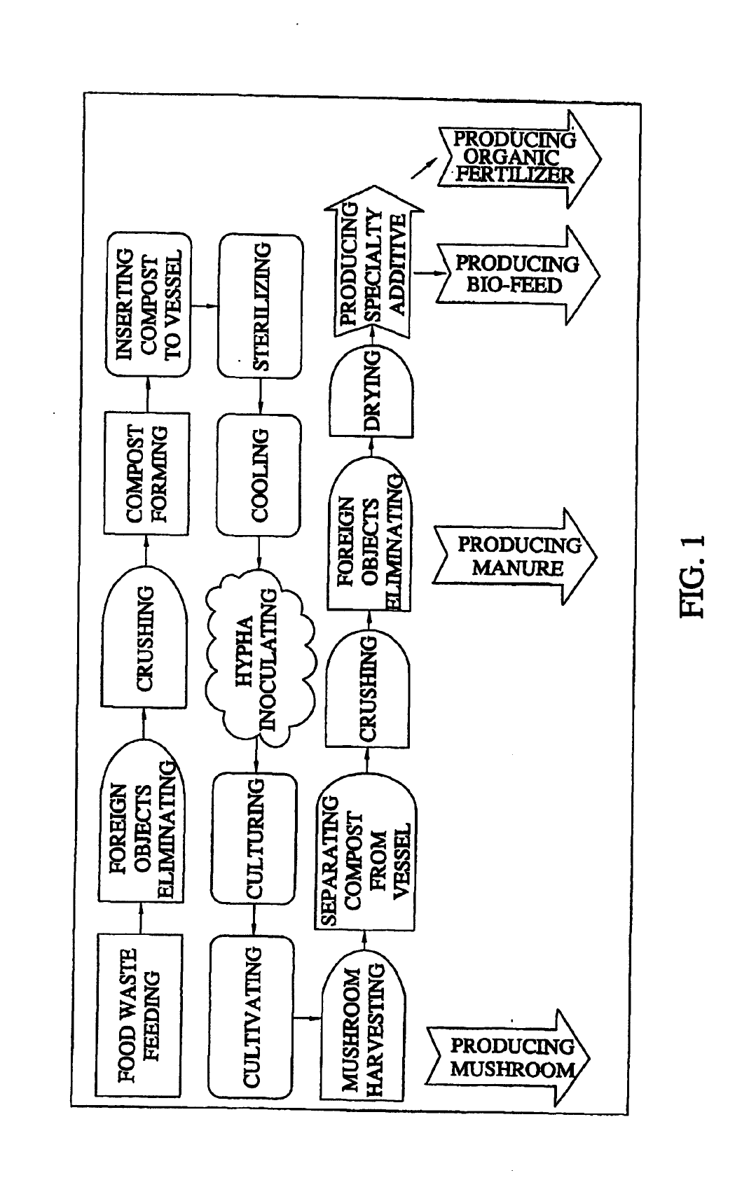

FIG. 2 is a table that shows a hyphae state of Pleurotus ostreatus DH-1012 and Pleurotus ostreatus Heukpyung cultured at different salt concentrations.

FIG. 3 is a schematic diagram of a food wastes pre-processing system.

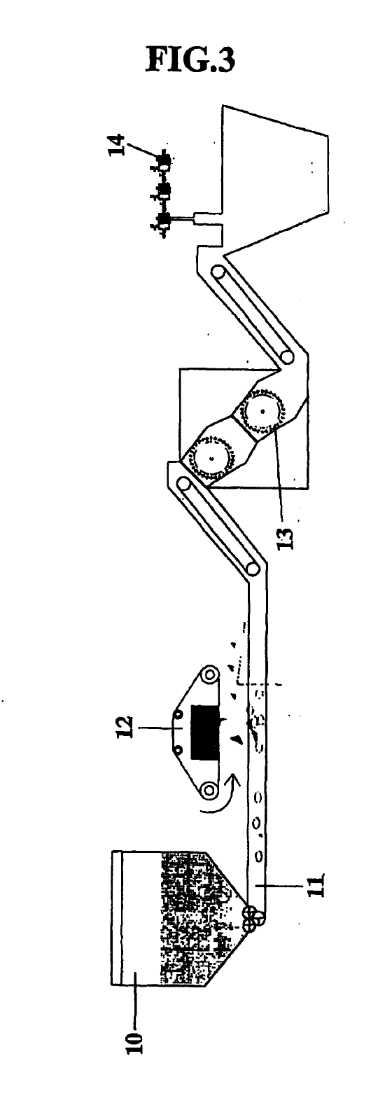

FIG. 4 is a schematic diagram of a specialty additive producing system using the compost remaining after harvesting mushroom.

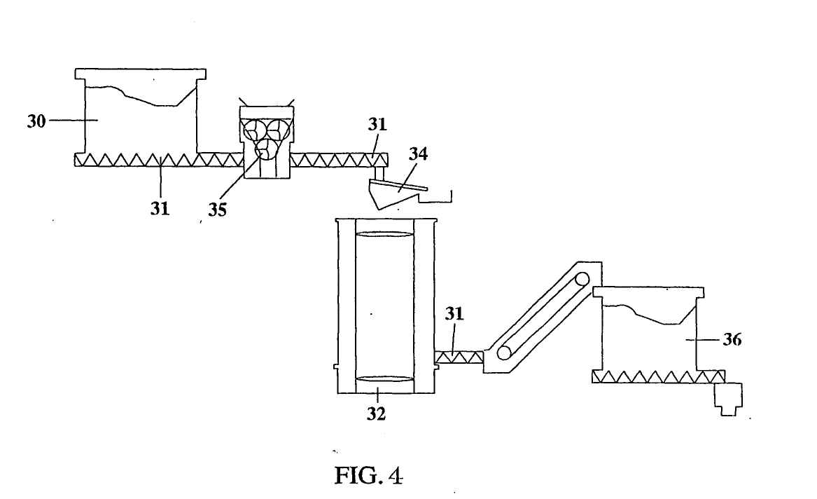

FIG. 5 is a structural diagram of a digital automatic monitoring and control system that shows a configuration of digital automatic monitoring and control of the whole process system according to the present invention.

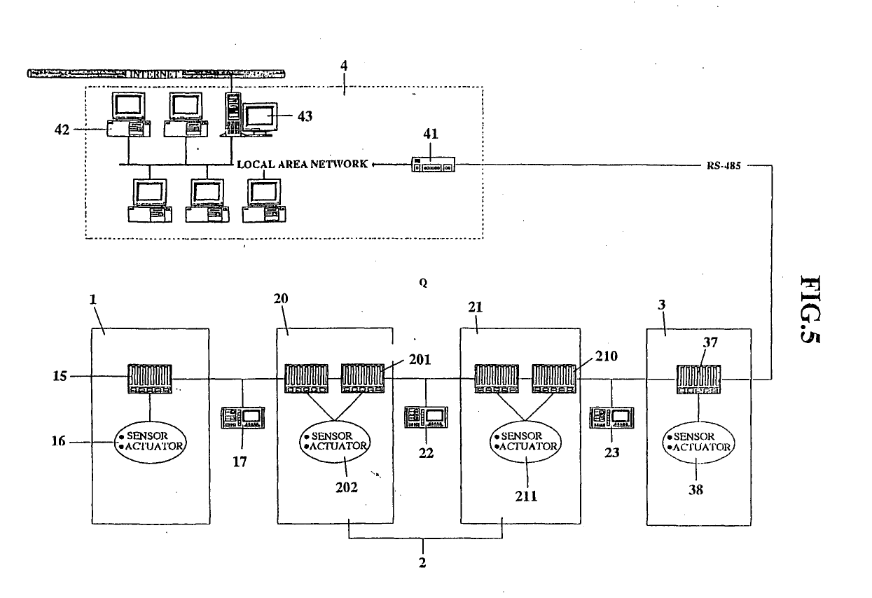

FIG. 6 is a design concept of a central console implementing the digital automatic monitoring and control system according to the, present invention.

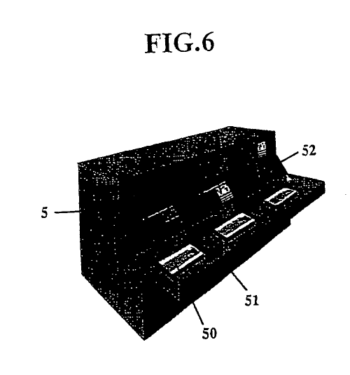

### BEST MODE FOR CARRYING OUT THE INVENTION

Hereinafter, preferred embodiments of the present invention will be described in detail with reference to the accompanying drawings.

Embodiment 1

The Method of Breeding Salt Tolerant Strains Pleurotus ostreatus DH-1012

First, 26 kinds of oyster pleurotus collected in Korea are numbered 1 to 26, and the 26 kinds are each tested in different growing conditions (optimum culturing temperature, culturing humidity, optimum growing temperature, humidity, etc.) to check the optimum growing conditions.

For the hyphae growing temperature, at first the hyphae were grown in the PDA composts with 5° C. variations in the temperature range of 10 to 35° C. which is a temperature range for growing oyster pleurotus. And, hyphae were again grown with 1° C. variations for testing an operation hyphae growing temperature in the best temperature range. Optimum growing temperature and humidity are tested in the same way as actual growing.

Next, to test the salt tolerant character, the hyphae were cultured in the PDA composts with increasing salt concentration by 0.5% respectively in the range of 0% to 3%, and the hypha, No.12 Pleurotus ostreatus which is less affected in growing by the increased salt concentration is selected and named Pleurotus ostreatus—12.

In an aseptic condition, monospores of the Pleurotus ostreatus—12 were separated by the general monospore separating method and thereby 76 strains were obtained and numbered No. 1 to 76. Then mutation of the No. 1 to 76 strain was induced by ultraviolet ray irradiation and the strain No.38 was selected as an auxotroph strain having fertility and named Pleurotus ostreatus—38.

In addition, from the oyster mushroom Pleurotus ostreatus Nonggi 2-1 which is a recommended cultivar in Korea and has high quality, monospores were separated. From which 82 strains were obtained and numbered NO. 1 to 82 and mutation induced by ultraviolet irradiation, and the strain No.16 was selected as an auxotroph strain having fertility and named Pleurotus ostreatus—16.

An oyster genus is tetraplar and dioecious, the number of its spores of basidium is 4, and it has 2 incompatible factors. A spore has one nucleus and clamp connector. Therefore, the oyster genus has 4 mating-types, and the two auxotroph strains have complementarity due to being binucleated by mating. By monospore mating between Pleurotus ostreatus—38 and Pleurotus ostreatus—16 which are selected by using such properties, an oyster mushroom strain Pleurotus ostreatus DH-1012 which is a somatic cell hybrid with salt tolerant capability and high quality.

Embodiment 2

The Characteristic of shapes, Physiology, and Culturing of Pleurotus ostreatus DH-1012

To study characteristics about the culturing and the forming of the fruit body of Pleurotus ostreatus DH-1012, obtained by the process in embodiment 1, Pleurotus ostreatus DH-1012 was tested in different growing conditions to check the optimum growing condition.

For the hyphae growing temperature, at first the hyphae were grown in the compost with 5° C. variations in the temperature range of 10 to 35° C. at which temperature the oyster genus (pleurotus) generally grows. And, the hyphae were again grown with 1° C. variations in the temperature range where the oyster genus grows best, for testing the optimum hyphae growing temperature. Optimum growing temperature and humidity are tested in the same way as actual growing.

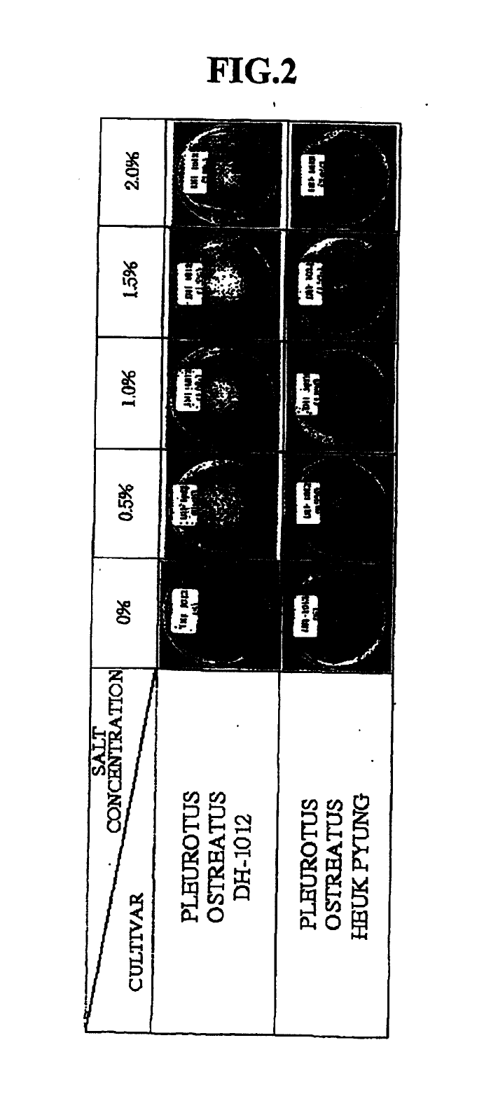

The tested salt tolerant property, the most important characteristics, of the Pleurotus ostreatus DH-1012 was compared with Pleurotus ostreatus Heukpyung, the type recommended by the office of Rural Development Administration, and which is recently cultivated most in Korean farms because of its high quality. As shown in FIG. 2, Pleurotus ostreatus Heukpyung did not grow well in the salty compost, but Pleurotus ostreatus DH-1012 grew well in the compost made of Korean food wastes whose slat concentration is 2%, and it can grow in the compost whose salt concentration is maximum 3%.

Also, the compost suitable for culturing the Pleurotus ostreatus DH-1012 was also tested. In general, edible mushrooms are grown in cultivating shelve, cultivating box, cultivating bottle filled with composts such as rice-straw, sawdust, and cotton generally used. The present invention was also tested in the above conditions and for the food wastes compost, the bottle cultivation is found to be suitable.

The shape of the Pleurotus ostreatus DH-1012 which is cultivated in the bottle like general edible mushrooms is described hereinafter. The pileus is gray-brown half-umbrella shape, the mushroom has bundle shape which appears as a bundle, the pileus is small, and stem is thick so that the quality is good. About 1.0 g per cultivating bottle of 850 cc are harvested, and weight of one mushroom is about 15 g. Frequent ventilation is required but the hyphae activity is very good, and the temperature suitable for hyphae growing is 20 to 23° C. The mushroom cultivating temperature is 8 to 18° C., and preferably little low temperature of 8 to 12° C. The time required for first development is in about 45 days.

The culturing and fruit body forming characteristics of Pleurotus ostreatus DH-1012 is tested for about 3 years more than 20 times, and the result shows that the hereditary characteristics are stable, and it is still cultivated.

The table 1 shows the comparison result of characteristics of Pleurotus ostreatus DH-1012 and Pleurotus ostreatus Heukpyung which is a recommended type by the office of Rural Development Administration.

TABLE 1

| | Hyphae | | | | | | | Yield | Salt |
|---|---|---|---|---|---|---|---|---|---|
| | Growing | Culturing | | Pileus | | Ventilation | | (g/850 cc | tolerant |
| Species | temperature | temperature | Humidity | shape | Color | frequency | Quality | bottle) | property |
| DH-1012 | 22˜25° C. | 8˜15° C. | 95% | Medium | Light- | Regular | Regular | 120 g | Good |
| | | | | | gray | | | | |
| Heukpyung | 25˜30° C. | 10˜16° C. | 90% | Medium | Black- | Frequent | Good | 80 g | poor |
| | | | | | brown | | | | |

Embodiment 3

The Oyster Mushroom Nutrition Analysis

Table 2, shows the result of comparison between the component of oyster mushroom cultivated in the cellulose compost consisting of 20% rice bran and 80% sawdust, and in the food waste compost consisting of 67% food waste and 33% sawdust. The oyster mushrooms cultivated in the cellulose compost and in the food waste compost have similar basic nutritional component but the mushroom cultivated in the food waste is richer in immune and carcinostatis functional substance like vitamin such as vitamin A and C, ergosterol, linoleic acid.

TABLE 2

| Component | Cellulose compost | Food waste compost |
|---|---|---|
| Moisture(%) | 84.8 | 84.4 |
| ASH(%) | 1.0 | 1.8 |
| Crude fat(%) | 0.1 | 0.1 |
| Crude protein(%) | 4.4 | 4.1 |
| Vitamin B1(mg/100 g) | 4.6 | 1.3 |
| Vitamin B2(mg/100 g) | 0.7 | 0.6 |
| Vitamin A(IU/100 g) | — | 4522.4 |
| Vitamin C(mg/100 g) | — | 3.0 |
| Salt(%) | 0.02 | 0.03 |
| Carbohydrate(%) | 3.3 | 3.9 |
| Calories(kcal) | 31.7 | 32.9 |
| Iron(mg/100 g) | 2.50 | 0.96 |
| Phosphorus(mg/100 g) | 92.9 | 141.4 |
| Ergosterol(IU) | 675.5 | 989.1 |
| Sodium(mg/100 g) | 8.16 | 29.9 |
| Linol acid(mg/100 g) | 107.89 | 128.98 |
| Niacin(mg/100 g) | 1.6 | 1.5 |
| Dietary fiber(%) | 6.4 | 5.7 |
| Potassium(mg/100 g) | 263.5 | 386.9 |

Embodiment 4

Specialty Biofeed Additive Chemical Analysis

After cultivating oyster mushrooms in the food waste compost consisting of 67% food waste and 33% sawdust and harvesting the mushrooms, the food waste compost is dried to the 13% water content, and used as a specialty additive for specialty bio-feed and organic fertilizer. As shown in table 3, large amount of immune and carcinostatis functional substances such as vitamin A, D, β-Glucan, ergosterol, linoleic acid, etc. are present in the compost.

TABLE 3

| Component | Bio-feed | Component | Bio-feed |
|---|---|---|---|
| Moisture(%) | 13.00 | ADF(%) | 49.26 |
| Crude protein(%) | 8.50 | NDF(%) | 65.57 |
| Crude fat(%) | 1.20 | Lignin(%) | 8.34 |
| Crude fiber(%) | 42.19 | Silica(%) | 0.85 |
| Crude ash(%) | 8.90 | NaCl(%) | 1.49 |
| Ca(%) | 2.37 | β-Glucan(%) | 3.92 |
| P(%) | 0.37 | Glucose(%) | 0.23 |
| K(%) | 0.43 | Sucrose | N.D |
| Mg(%) | 0.11 | Galactose | N.D |
| Na(%) | 0.67 | Mannose | N.D |
| Zn(ppm) | 33.48 | Xylose | N.D |
| Fe(%) | 0.13 | Arabinose | N.D |
| Mn(ppm) | 27.48 | Ergosterol(ppm) | 323.08 |
| Cu(ppm) | 4.05 | Vitamin A(IU/kg) | 103.71 |
| Co(ppm) | 0.51 | Vitamin B₁ | N.D |
| S(%) | 0.11 | Vitamin B₂ (ppm) | 37.98 |
| Al(%) | 0.16 | Vitamin C | N.D |
| Si(%) | 0.40 | Vitamin D(IU/kg) | 53,772.90 |
| Pb(ppm) | 4.98 | Vitamin E | N.D |
| Cd | N.D | Aflatoxin | N.D |
| | (Not Detected) | | |
| As(ppm) | 0.08 | Amino acid | 0.56 |
| | | composition | |
| | | Aspartic Acid | |
| Hg | N.D | Threonine | 0.29 |
| Cr(ppm) | 1.84 | Serine | 0.29 |
| F | N.D | Glutamic Acid | 0.77 |
| Proline | 0.27 | Histidine | 0.29 |
| Glycine | 0.32 | Lysine | 0.24 |
| Alanine | 0.40 | Arginine | 0.27 |
| Valine | 0.32 | Cystine | 0.13 |
| Isoleucine | 0.29 | Methionine | 0.08 |
| Leucine | 0.43 | Tryptophan | 0.03 |
| Tyrocine | 0.35 | Fatty Acids | 0.51 |
| | | composition | |
| | | Myristic acid C_{14:0} | |
| Phenylalanine | 0.35 | Pentadecanoic acid | 0.43 |
| | | C_{15:0} | |
| Palmitic acid C_{16:0} | 20.10 | Arachidic acid C | 0.61 |
| | | C_{20:0} | |
| Palmiltoleic acid | 0.70 | Eicosatrienoic acid | 0.50 |
| C_{16:1} | | C_{20:3n6} | |
| Magaric acid C_{17:0} | 0.69 | Arachidonic acid | 0.82 |
| | | C_{20:4n3} | |
| Stearic acid C_{18:0} | 18.07 | EPA C_{20:5n3} | 1.22 |
| Oleic acid C_{18:1} | 16.92 | Behenic acid C_{22:0} | 0.80 |
| Lonoleic acid C_{18:2n6} | 15.26 | Docosatetraenoic | 2.34 |
| | | acid C_{22:4n6} | |
| Linolenic acid | 0.60 | DPA C_{22:5n3} | 0.59 |
| C_{18:3n3} | | | |
| Nonadecanoic acid | 0.63 | Unknown | 19.21 |
| C_{19:0} | | | |

Embodiment 5

Organic Fertilizer Additive Physiochemical Analysis

After cultivating oyster mushrooms in the food waste compost consisting of 67% food-waste and 33% sawdust and harvesting the mushrooms, the food waste compost is dried to the 13% water content, and used as a organic fertilizer and organic fertilizer additive. The same sample in Table 3 was used for organic fertilizer additive physiochemical analysis as shown in tables 3 & 4, large amount of immune and carcinostatis functional substances and necessary element for organic fertilizer such as N, P, K, and organic matter are present in the compost. The pH, and electric conductivity of the compost is suitable for organic fertilizer.

TABLE 4

| Component | | Unit | Result | Remark |
|---|---|---|---|---|
| Moisture | | % | 13.0 | |
| Total Nitrogen | | % | 0.87 | |
| Organic Matter | | % | 62.83 | Dry matter base |
| Electric Conductivity | | ms/cm | 2.38 | |
| Cation Exchange | | meq/100 g | 5.70 | Dry matter base |
| Capacity | | | | |
| Ammonia-Nitrogen | | ppm | 133.54 | |
| Nitrate-Nitrogen | | | — | |
| Available | | ppm | 496.87 | |
| Phosphovs(P₂O₅) | | | | |
| Volume Density | | g/l | 377.79 | |
| Volume Density | | g/l | 217.14 | Dry matter base |
| Water Holding Capacity | | % | 381.72 | |
| Carbon/Nitrogen Ratio | | | 48.09 | |
| Particle Density | | | 0.2343 | |
| pH(1:5) | | | 6.76 | |
| Exchangeable | Ca | meq/100 g | 12.32 | |
| | K | meq/100 g | 12.48 | |
| | Mg | meq/100 g | 7.56 | |
| | Na | meq/100 g | 31.14 | |

| | | |
|---|---|---|
| Cu | ppm | 6.69 |
| Pb | ppm | 2.18 |
| Cr | ppm | 8.19 |
| Hg | | — |
| As | ppm | 0.05 |
| Cd | ppm | 0.65 |
| NaCl | % | 0.41 |

Embodiment 6

Organic Fertilizer Government Regulatory Standards Comparison Analysis

Table 5 shows the result of comparing items of food waste oyster mushroom compost with the government regulatory standards for certifying suitability of the organic fertilizer of the present invention. As shown in table 5, the food waste compost satisfies all the regulatory standards except salt ratio. As for the salt ratio, it is 1.49%, which is larger than the regulatory standards 1%, but the organic fertilizer additive of the present invention is mixed with other organic fertilizers to be used as specialty organic fertilizer, so that the salt ratio is adjustable.

TABLE 5

| | Ministry of | Ministry of | Food waste Oyster |
|---|---|---|---|
| | Agriculture | Environment | Mushroom |
| Items | Standards | Standards | Compost |
| Cu | <500 mg/kg | <500 mg/kg | 6.69 mg/kg |
| Pb | <150 mg/kg | <150 mg/kg | 4.98 mg/kg |
| Cr | <300 mg/kg | <300 mg/kg | 8.19 mg/kg |
| Hg | <2 mg/kg | <2 mg/kg | Not Detected |
| As | <50 mg/kg | <50 mg/kg | 0.08 mg/kg |
| Cd | <5 mg/kg | <5 mg/kg | 0.65 mg/kg |
| NaCl | <1% | <1% | 1.49% |
| CN Ratio* | | <50 | 48.09% |
| Organic Matter | | >25% | 62.83% |

*CN ratio: Carbon/Nitrogen ratio

The novel strain Pleurotus ostreatus DH-1012 in accordance with the invention has the salt-tolerant capability and produces high quality mushroom.

In addition, if salt-tolerant mushroom strains including the novel strain Pleurotus ostreatus DH-1012 are used, environment-friendly treatment of the food wastes can be implemented without causing secondary environmental pollution, while keeping sustainable development of natural ecological system.

In addition, the method for disposing the food wastes and the system of pre-processing it using the salt tolerant mushroom strains in accordance with the invention achieves primary effect such as treatment of the food wastes at a low cost and environment-friendly treatment. In particular, mushrooms can be harvested and the mushroom hyphae in the compost remaining after harvesting the composts decompose any compounds in the mixture and foreign objects such as toothpicks, wooden chopsticks, and papers. The mushroom hyphae spread in the mixture results in the specialty additive for specialty bio-feed and organic fertilizer and manure with rich organic and inorganic objects and of a high rate of intestinal absorption. Thus in accordance with the present invention, the enhanced economical effects by the specialty bio-feed and organic fertilizer from the compost remaining after harvesting the mushrooms as well as by cultivated mushrooms can be achieved. The Office of Rural Development Administration noted that the mushroom is the most profitable agricultural product per unit area in Korea.

The invention is provided with automatic equipment and an digital automatic monitoring and control system that can treat large amount of food wastes and employs the concept of automatic control design for cultivating mushroom and producing specialty additive for bio-feed and organic fertilizer and manure, so that the invention contributes to recycling the food wastes as economical resources with high profitability by improving specialty bio-feed and organic fertilizer as well as cultivated mushroom quality and yields thereof.

The present invention has been described in detail. However, it should be understood that the detailed description and specific examples, while indicating preferred embodiments of the invention, are given by way of illustration only, since various changes and 1-5 modifications within the spirit and scope of the invention will become apparent to those skilled in the art from this detailed description.

1. A novel salt tolerant oyster mushroom strain Pleurotus ostreatus DH-1012 (deposit No. KCTC 0938BP)

2. A method of producing a novel salt tolerant oyster mushroom strain Pleurotus ostreatus DH-1012, comprising the steps of:
1) culturing numbers of hyphae in the composts with different salt concentration and selecting hyphae that are less affected by higher salt concentrations in growing;
2) obtaining numbers of strains by separating monospores from said selected hyphae in an aseptic condition and then inducing mutation by irradiating ultraviolet rays;
3) selecting an auxotroph strain having fertility among said mutation induced strains;
4) obtaining numbers of strains by selecting one among high quality oyster mushrooms and separating monospores therefrom, then inducing mutation by irradiating ultraviolet rays;
5) selecting an auxotroph strain having fertility among said mutation induced strains; and
6) producing a somatic cell hybrid strain by monospore mating between the strain selected in step 3) and the strain selected in step 5).

3. The method according to claim 2, wherein said strain used in step 1 is Pleurotus ostreatus.

4. The method according to claim 2, wherein said strain used in step 4 is Pleurotus ostreatus Nonggi 2-1.

5. The method according to one of the claims 2 to 4, wherein in the step 1, the hyphae are cultured in the PDA composts while varying the salt concentration by 0.5% within the range of 0-3%, and a hyphae the growth of which is less affected by the variation/increment of salt concentration is selected.

6. A method of disposing food wastes using salt tolerant mushroom strains, comprising the steps of:
a food wastes pre-processing step for forming a suitable compost for mushroom cultivating; and
a mushroom cultivating step for cultivating mushrooms by inoculating salt tolerant mushroom strains into said compost and culturing the mushrooms.

7. A method according to claim 6, further comprising a specialty additive producing step for producing specialty additive, which is used for mixing with other feeds and organic fertilizers, with the compost remaining after harvesting said cultivated mushrooms.

8. A method according to claim 6, further comprising a manure producing step for producing manure with the compost remaining after harvesting said cultivated mushrooms.

9. The method according to claim 6, wherein said salt tolerant oyster mushroom strain is a novel strain Pleurotus ostreatus DH-1012.

10. The method according to claim 6, wherein said food wastes pre-processing step comprises the steps of:
1) eliminating foreign objects from food wastes;
2) crushing said food wastes from which the foreign objects have been eliminated into a predetermined size; and
3) deodorizing said crushed food wastes, wherein each of said steps can be performed simultaneously or sequentially.

11. The method according to claim 6, wherein said mushroom cultivating step comprises the steps of:
1) forming a compost by adjusting water content of the crushed food wastes from which the foreign objects have been eliminated;
2) inserting a predetermined amount of said formed compost into a cultivating vessel;
3) sterilizing said compost inserted into the cultivating vessel;
4) cooling said sterilized compost, then inoculating salt tolerant mushroom strains into said cooled compost;
5) culturing said inoculated salt tolerant strains; and
6) cultivating the mushroom strains and harvesting said cultivated mushrooms.

12. The method according to claim 7, wherein said specialty additive producing step comprises the steps of:
1) separating the remaining compost after harvesting mushroom from the cultivating vessel;
2) crushing the separated compost into a predetermined size;
3) eliminating foreign objects from the crushed compost; and
4) producing specialty additive containing mushroom hyphae by drying said compost from which foreign objects have been eliminated.

13. The method according to claim 8, wherein said manure producing step comprises the steps of:
1) separating the remaining compost after harvesting mushroom from the cultivating vessel;
2) crushing the separated compost into a predetermined size; and
3) eliminating foreign objects from the crushed compost.

14. The method according to one of the claims 6 to 12, wherein said food wastes pre-processing step comprises the steps of eliminating foreign metallic objects from food wastes, crushing said food wastes from which foreign objects have been eliminated into a predetermined size, and deodorizing said crushed food wastes,
wherein said mushroom cultivating step comprises the steps of:
forming a compost by mixing dry cellulose with food wastes so that the water content of the crushed food wastes from which foreign objects have been eliminated is 50 to 70%;
inserting a predetermined amount of said formed compost into the cultivating vessel;
sterilizing said compost by supplied steam from the boilers at 60° C. to 121° C. for 1 to 5 hours;
cooling said compost down to 20 to 25° C.;
inoculating a predetermined amount of the novel strain Pleurotus ostreatus DH-1012 into said compost;
culturing the strain for 15 to 30 days in an aseptic culturing room at 20 to 25° C.;
cultivating the cultured strain at 8 to 18° C.; and
harvesting the mushroom;
and in that said specialty additive producing step comprises the steps of:
separating the compost remaining after harvesting the mushrooms from the cultivating vessel;
crushing said separated compost into a predetermined size;
eliminating foreign objects from said crushed compost; and
producing specialty additive by drying said foreign objects eliminated compost so that the water content of the compost is 10 to 15%.

15. The method according to one of the claims 6 to 13, wherein said food wastes pre-processing step comprises the steps of eliminating foreign metallic objects from food wastes, crushing said food wastes from which foreign objects have been eliminated into a predetermined size, and deodorizing said crushed food wastes,
wherein said mushroom cultivating step comprises the steps of:
forming a compost by mixing dry cellulose with food wastes so that the water content of the crushed food wastes from which foreign objects have been eliminated is 50 to 70%;
inserting a predetermined amount of said formed compost into the cultivating vessel;
sterilizing said compost by supplied steam from the boilers at 60° C. to 121° C. for 1 to 5 hours;
cooling said compost down to 20 to 25° C.;
inoculating a predetermined amount of the novel strain Pleurotus ostreatus DH-1012 into said compost;
culturing the strain for 15 to 30 days in an aseptic culturing room at 20 to 25° C.;
cultivating the cultured strain at 8 to 18° C.; and
harvesting the mushroom;
and in that said manure producing step comprises the steps of:
separating the compost remaining after harvesting the mushrooms from the cultivating vessel;
crushing said separated compost into a predetermined size; and
eliminating foreign objects from said crushed compost.

16. A mushroom cultivated according to the method of one of the claims 6 to −11.

17. A specialty bio-feed and organic fertilizer additive produced according to the method of one of the claims 6 to 12.

18. A specialty bio-feed produced by mixing with the specialty additive of claim 17.

19. A specialty organic fertilizer produced by mixing with the specialty additive of claim 17.

20. Manure produced according to the method of one of the claims 6 to 13.

21. A system of disposing food wastes using salt tolerant mushroom strains, comprising:
a food wastes pre-processing system for forming a suitable compost for cultivating mushrooms; and
a mushroom cultivating system for cultivating mushrooms by inoculating salt tolerant mushroom strains into said compost and culturing the mushroom strain in the compost.

22. A system according to claim 21 further comprising a specialty additive producing system for producing specialty additive, which is used for mixing with specialty bio-feed and organic fertilizer, with the compost remaining after harvesting said cultivated mushrooms.

23. A system according to claim 21 further comprising manure producing system for producing manure with the compost remaining after harvesting said cultivated mushrooms.

24. The system according to claim 21, wherein said salt tolerant oyster mushroom strain is a novel strain Pleurotus ostreatus DH-1012.

25. The system according to claim 21, wherein said food wastes pre-processing system comprises an entrance through which the food wastes enter and an exit through which the food wastes are discharged, a fixed blade which is fixed to a frame and has a plurality of toothed blades and a rotating blade which is installed in a predetermined gap from the fixed blade between said entrance and said exit, and at least one crusher for crushing the food wastes into a predetermined size with the rotation of said rotating blade and at least one metallic foreign objects eliminating means,
wherein said crusher and said foreign objects eliminating means are designed air-tight closed system, and the inner space sealed by said air-tight closed system is communicated with at least one location by means of stench gas suction pipes to bio-deodorizing system.

26. The system according to claim 21, wherein said mushroom cultivating system comprises:
at least one compost forming means for forming a compost by adjusting water content of the crushed food wastes;
at least one compost inserting means for inserting a predetermined amount of said compost into a cultivating vessel;
at least one sterilizing means for sterilizing the compost inserted in the cultivating vessel;
at least one strain inoculating means for cooling said sterilized compost and inoculating salt tolerant mushroom strains into said compost;
at least one hyphae culturing means for culturing said inoculated salt tolerant mushroom strains; and
at least one mushroom cultivating means for cultivating mushrooms with said cultured hyphae.

27. The system according to claim 26, wherein said compost forming means comprises a transporting means for transporting said food wastes pre-processed in said food wastes pre-processing system, and a mixer for mixing dry cellulose with said food wastes that are transported by said transporting means and for adjusting the water content thereof.

28. The system according to claim 26, wherein said compost inserting means comprises a transporting means for transporting said compost formed by said compost forming means, a supplying means for supplying the cultivating vessel for inserting said transported compost therein, an inserter for inserting said transported compost into said supplied cultivating vessel, and a capping means for capping said vessel.

29. The System according to claim 26, wherein said compost sterilizing means thereof comprises a transporting means for transporting said cultivating vessel with the compost inserted therein, and a sterilizer for sterilizing said cultivating vessel transported by said transporting means.

30. The system according to claim 26, wherein said strain inoculating means comprises a transporting means for transporting said cultivating vessel into which the sterilized compost is inserted, a means for cooling said cultivating vessel with the compost inserted therein and maintaining an aseptic condition, and an inoculater for inoculating a predetermined amount of salt tolerant mushroom strains into said cooled compost.

31. The system according to claim 26, wherein said hyphae culturing means comprises a transporting means for transporting said strain-inoculated cultivating vessel, and a cooler/heater, a humidifier, a means of illumination, and a ventilation system for making an optimum culturing condition for said inoculated salt tolerant mushroom strain in said transported cultivating vessel.

32. The system according to claim 26, wherein said mushroom cultivating means thereof comprises a transporting means for transporting said cultured hyphae, and a cooler/heater, a humidifier, a means of illumination, and a ventilation system for making an optimum culturing condition for the cultured salt tolerant mushroom hyphae in said transported cultivating vessel.

33. The system according to claim 22, wherein said specialty additive producing system comprises:
a compost separating means for separating the compost from the cultivating vessel after harvesting mushrooms;
a crushing means for crushing said separated compost into predetermined size:
a foreign object separating means for eliminating foreign objects from said crushed compost;
a drying means for drying said foreign objects eliminated compost; and
a packing means for packing said dried specialty additive.

34. The system according to claim 33, wherein said compost separating means of said specialty additive producing system comprises a transporting means for transporting said cultivating vessel in which only the compost is left after the cultivated mushrooms, are harvested, a separator for separating the compost from said transported cultivating vessel, and a storing tank for storing said separated compost.

35. The system according to claim 33, wherein said drying means of said specialty additive producing system comprises a transporting means for transporting said separated compost to a feeding hopper, an inputting means for inputting said transported compost to a dryer in a predetermined amount, and an indirect drying means using heater or steam from a boiler or a direct drying means using a hot air blower.

36. The system according to claim 33, wherein said foreign objects eliminating means of said specialty additive producing system comprises a transporting means for transporting the dried compost, an eliminating means for eliminating foreign objects from said transported compost, and a discharging means for discharging said eliminated foreign objects.

37. The system according to claim 33, wherein said packing means of said specialty additive producing system comprises a transporting means for transporting said specialty additive crushed into a predetermined size by said crushing means, a storing means for storing said specialty additive transported by said transporting means, a means for providing a packing case for packing said stored specialty additive, and a packing device for packing said specialty additive which is transported from said storing means in a predetermined amount into said packing case which is provided by said providing means.

38. The system according to claim 23, wherein said manure producing system comprises:
a compost separating means for separating the compost from the cultivating vessel after harvesting mushrooms;
a crushing means for crushing said separated compost into predetermined size:
a foreign object separating means for eliminating foreign objects from said crushed compost.

39. The system according to claim 38, wherein said compost separating means of said manure producing system comprises a transporting means for transporting said cultivating vessel in which only the compost is left after the cultivated mushrooms are harvested, a separator for separating the compost from said transported cultivating vessel.

40. The system according to claim 38, wherein said foreign objects eliminating means of said manure producing system comprises a transporting means for transporting the dried compost, an eliminating means for eliminating foreign objects from said transported compost, and a discharging means for discharging said eliminated foreign objects.

41. The system according to one of the claims 21 to 40, wherein said transporting means is a horizontal belt conveyor, wherein said horizontal belt conveyor is made of elastic material so that the belt conveyor is stretched by the weight of the material transported by the conveyor while both ends of said conveyor are held at a predetermined height by a shape fixing guide.

42. The system according to claim 41, wherein said conveyor is a screw conveyor having a rotary shaft driven by the power of a transporting motor and spiral blade members fisted to the rotary shaft.

43. The system according to one of the claims 21 to 40, further comprising a digital monitoring and control system for automatically monitoring and controlling the food wastes pre-processing system, the mushroom cultivating system, the specialty additive producing system, the manure producing system.

44. The system according to claim 43, wherein the digital monitoring and control system comprises:
a central console for monitoring and controlling the food wastes pre-processing system, the mushroom cultivating system, the specialty additive producing system, the manure producing system processes performed by the systems and products produced by the systems through the input/output and transmission/reception of various monitoring and control data:
local control boards installed in the systems and controlled by said central console;
sensors installed in each component of said systems, provide signals to local control board and central consol, and linked to said central consol; and
a communication interface means performing transmission and reception of data between the central console and the local control boards or between the central console and the sensors.

45. Mushroom cultivated by the system according to one of the claims 21 to 40.

46. Specialty bio-feed and organic fertilizer additive produced by the system according to one of the claims 21 to 40.

47. A specialty bio-feed produced by mixing with the specialty additive of claim 46.

48. A specialty organic fertilizer produced by mixing with the specialty additive of claim 46.

49. Manure produced according to the method of one of the claims 21 to 40.
